(19) United States
(12) Patent Application Publication
(10) Pub. No.: US 2020/0015872 A1
(43) Pub. Date: Jan. 16, 2020

(54) Method for Producing an Osteosynthetic Implant, and Bone Nail

(71) Applicant: GLW, Inc., Waxhaw, NC (US)

(72) Inventor: Christian Lutz, Heikendorf (DE)

(73) Assignee: GLW, Inc., Waxhaw, NC (US)

(21) Appl. No.: 16/582,323

(22) Filed: Sep. 25, 2019

Related U.S. Application Data
(63) Continuation of application No. 15/128,679, filed on Sep. 23, 2016.

(30) Foreign Application Priority Data
Mar. 24, 2014 (DE) 10 2014 103 972.6

(51) Int. Cl. A61B 17/84; A61B 17/72

(52) U.S. Cl. CPC A61B 17/846; A61B 17/72

(57) ABSTRACT

The invention relates to a method for producing an osteosynthetic implant, comprising the steps of producing a load-bearing metal component and coating the load-bearing component with a biocompatible material.

### CROSS-REFERENCE TO RELATED APPLICATIONS

This application is a continuation of U.S. Non-Provisional patent application Ser. No. 15/128,679, filed on Sep. 23, 2016, which is a 371 application of PCT Application No. PCT/DE2015/100109, filed Mar. 17, 2015. Each of these related applications is incorporated by reference into this disclosure in its entirety.

### FIELD

The invention relates to a method for producing an implant for osteosynthesis. The invention also relates to a bone nail.

### DETAILED DESCRIPTION OF SELECTED EXAMPLES

In traumatology used implants for osteosynthesis are known for example from EP 1488752 A1. The partially very laborious and time-consuming machining processes for the production of the body-contoured shape and the load-bearing function prove to be disadvantageous. These procedures require the appropriate times in the manufacturing and complex and time-consuming process in quality control. Although these methods have been largely optimized in the last ten years, they now offer very little potential for reducing labor and thus also costs. The object of the invention is therefore to provide a method for producing an implant for osteosynthesis, which requires hardly any time- and labor-consuming manufacturing techniques, resulting in a relatively inexpensive and at the same time high-quality product. This object is achieved by the method having the features of claim 1 and the bone nail with the features of claim 10. The dependent claims respectively reflect advantageous embodiments of the invention.

It is the basic idea of the invention to produce the load-bearing part of an implant by a mass production process such as extrusion, stamping, forging or casting of a suitable profile or blank. In doing so the formfitting or tapered fit shape of the implant shall be made by injection molding of the profile/blank with an implantable plastic. The disadvantageous properties of a process are thereby offset by the advantages of the other method: a plastic usable in injection molding or extrusion alone would not possess the sufficient strength and would break, whereas a low manufactured profile would not have the formfitting shape and would therefore be incompatible for the patient. Both methods together, however, are relatively simple, inexpensive to use and fulfill the combined tasks of loading, and the biocompatibility. The inventive method for producing an implant for osteosynthesis does thus essentially consist of the steps of producing a load-bearing component of metal and coating of the load bearing component with a biocompatible material.

The coating is preferably carried out by injection molding. Alternatively, the coating may be made by extrusion.

As an alternative to complete injection of the load carrying element the load-bearing component cannot be covered completely, but only in sections. Thus, it is possible to provide an implant for osteosynthesis, that consists of a metallic section and a segment made of plastic. The metallic section must continue to be made of a high-quality metal, wherein the production of a plastic section continues to be able to save costs.

The production of the load carrying element does preferably take place as far as possible by means of extrusion molding or punching, wherein the used metal is preferably steel. Steel has the advantage that it is less sensitive to notches than titanium and the procurement is cheaper. Due to the higher rigidity and better formability of steel, it is possible to generate a profile that achieves the same stiffness as a titanium implant, but it requires less material.

However, the load-bearing component can also—if this can be done with little effort—be prepared by means of machining methods.

The metal used may also be a cobalt-molybdenum alloy, in particular a chromium-cobalt-molybdenum alloy. Finally, the metal used may also be titanium.

The biocompatible material, however, is preferably a polymer. More preferably, the polymer is selected from the group of polymers consisting of polyetheretherketones (PEEK) with carbon fiber reinforced polyetheretherketones and polyamides (PA).

Alternatively, also titanium can be used as a biocompatible material.

In any case, the outer shape of the implant is determined by the load-bearing component coated biocompatible material so that molding or extrusion substantially determines the shaping of the implant. It can therefore be possible that the outer shape of the implant is formed exclusively by the external shape of the biocompatible material, i.e. the outer shape of the load bearing component and the outer shape of the biocompatible material are dissimilar to one another.

According to another particularly preferred embodiment of the invention, the biocompatible material is continued to be designed as a drug carrier for medicines in particular for the prevention of infection or for accelerating bone growth.

The implant is preferably a bone or intramedullary pin or a bone plate.

Specifically, it is a bone nail for osteosynthesis, with a metal inlay, which is designed as a load-bearing component, and one by covering, in particular by injection molding or by extrusion of at least one portion of the metal inlay with the metal inlay connected plastic body, which substantially determines the outer shape of the bone nail. The bone nail is especially prepared by the inventive process.

According to the invention, the bone nail does preferably show an introduced groove in the covered section of the metal inlay transversely to its longitudinal axis. This groove, into which the plastic body comes in is used to fix the plastic body at the metal inlay. The groove is in particular for the metal inlay designed in circular design, so that the plastic body is secured against axial slipping at the metal inlay.

After to a further preferred embodiment, the bone nail shows a stopper axially extending from the metal inlay, which supports the plastic element at the metal inlay. The scope of the stopper can in particular also be used for a better access to the metal inlay during injection molding or extrusion, so that the metal inlay can be clamped in the corresponding device at the stopper—the outer circumference of the stopper will not be covered.

In particular, it is also provided that the covered section of the metal inlays is at least conical in sections. By this a very uniform force transmission from the metal insert into the plastic body is ensured.

After to a further preferred embodiment, the covered section of the metal inlays shows a thread, wherein the thread root and the thread crests are in particular rounded in order to avoid an impairing notch effect of the plastic body.

In particular, the thread may be penetrated by at least one axially extending groove, wherein preferentially three of such grooves are provided at an angle of 120.degree. to each other. This configuration allows a particularly stable connection of metal inlay and plastic body.

Finally, the bone nail may have a covered second metal inlay with an opening for receiving a locking screw in the distal area of the plastic body.

### DESCRIPTION OF FIGURES

The invention will be explained in more detail as per some examples. It is shown:

FIG. 1 a cross section of a bone nail according to a first exemplary embodiment;

FIG. 2 a cross section of a bone nail according to a second exemplary embodiment;

FIG. 3 a cross section of a bone nail according to a third exemplary embodiment;

FIG. 4 a cross section of a bone nail according to a fourth exemplary embodiment.

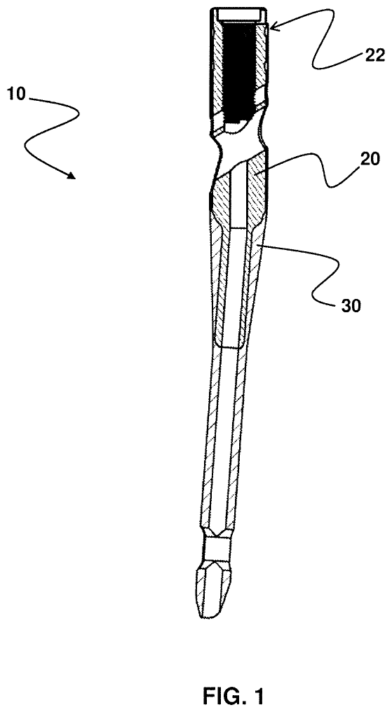

FIG. 1 shows a cross section of a bone nail (or intramedullary pin) according to a first embodiment. The bone nail 10 corresponds in its outer shape substantially to the shape of conventional bone or intramedullary nails.

Bone nail 10 does inventively consist of one as a load-bearing component formed metal inlay 20 and one with this connected plastic body 30 of a biocompatible polymer.

With this first embodiment, it is specifically provided that the entire surface with the exception of the proximal end side of the bone nail 10—is completely coated with the biocompatible material.

The metal inlay 20 shows in its proximal area two the metal inlay 20 circulating grooves 22, in which the plastic body 30 grips in and is therefore secured against slipping relative to the metal inlay 20.

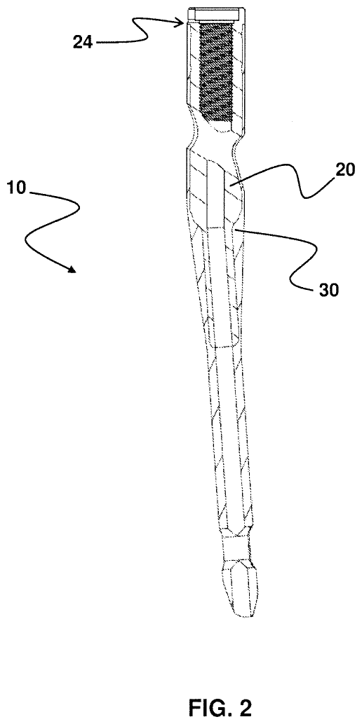

FIG. 2 shows a cross section of a bone nail according to a second embodiment. Bone nail 10 shown in FIG. 2 corresponds essentially to the embodiment shown in FIG. 1 with the difference that in addition a stopper 24 limiting the axial extent of the plastic body 30 is provided which extends radially from the metal-inlet 20.

The lateral surface of the stopper 24 and the surface of the plastic body 30 are formed in alignment, so that stopper 24 of the metal inlays 20 and the plastic body 30 are sealing against one another.

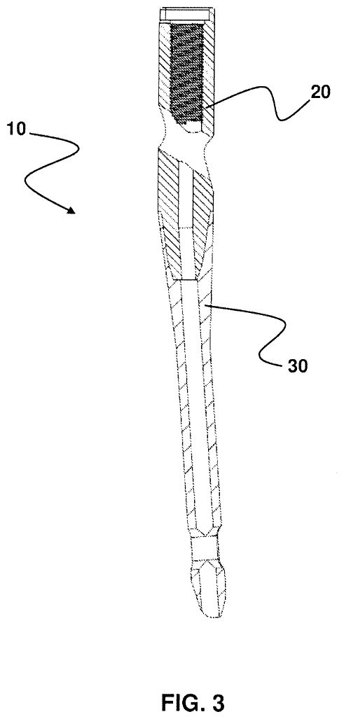

FIG. 3 shows a cross section of a bone nail according to a third embodiment. The metal inlay 20 is in its distal section conical, so that hereby a very uniform force transmission is achieved from the metal insert 20 into the plastic body 30.

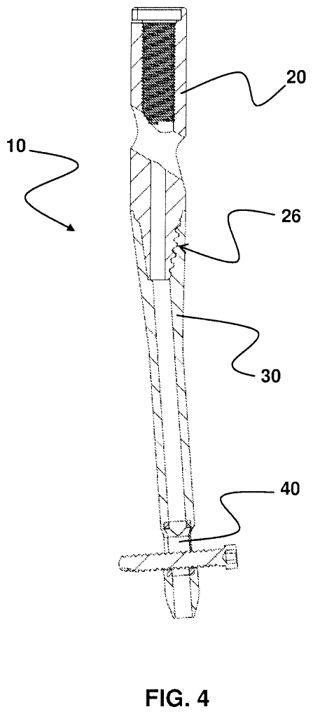

Finally, FIG. 4 shows a bone nail according to a fourth embodiment. The bone nail 10 according to the fourth embodiment displays the special feature of a metal inlay 20 whose distal conical section is provided with a thread 26 for a better connection to the plastic body.

In addition, this bone nail 10 shows the special feature of a second metal inlay 40 coated in the distal area of the plastic body 30, which is gripped by a locking screw. This additional metal insert provides a good interface for the distal metallic locking screw.

I claim:

1. A bone nail for osteosynthesis, comprising:
a first metal inlay comprising a load-bearing component having a proximal end, a distal end, a conical section disposed between the proximal end and the distal end, and a longitudinal axis, the conical section defining a thread;
a plastic body having a proximal area and a distal area, the proximal area disposed over the thread and connected to the first metal inlay; and
a second metal inlay disposed in the distal area of the plastic body and spaced from the first metal inlay, the second metal inlay defining an opening extending through the distal area of the plastic body along an axis disposed at an angle to the longitudinal axis and adapted to receive a locking screw.

2. The bone nail of claim 1, wherein the first metal inlay defines a groove disposed transverse to the longitudinal axis.

3. The bone nail of claim 2, wherein the groove comprises a circumferential groove.

4. The bone nail of claim 3, wherein the groove is disposed between the proximal end and the conical section.

5. The bone nail of claim 1, wherein the first metal inlay defines an axially-extending groove that penetrates the thread.

6. The bone nail of claim 5, wherein the first metal inlay defines three axially-extending grooves; and
wherein each axially-extending groove of the three axially-extending grooves penetrates the thread.

7. The bone nail of claim 6, wherein the three axially-extending grooves are provided at an angle of 120° to each other.

8. The bone nail of claim 1, wherein the proximal end of the first metal inlay defines a stopper that extends radially from the first metal inlay and limits the axial extent of the plastic body.

9. The bone nail of claim 8, wherein the stopper defines a lateral surface, and wherein the lateral surface is aligned with and sealing against the plastic body.

10. The bone nail of claim 1, wherein the first metal inlay comprises titanium.

11. The bone nail of claim 1, wherein the plastic body comprises a polymer.

12. The bone nail of claim 11, wherein the plastic body comprises a composition containing the polymer; and
wherein the polymer is selected from a group consisting of polyetherether ketones with carbon fiber reinforced polyetherether ketones and polyamides.

13. The bone nail of claim 1, wherein the conical section comprises a first conical section;
wherein the first metal inlay has a second conical section disposed between the proximal end and the distal end; and
wherein the plastic body is disposed over the first conical section and the second conical section.

14. A bone nail for osteosynthesis, comprising:
a first metal inlay comprising a load-bearing component having a proximal end, a distal end, a conical section disposed between the proximal end and the distal end, and a longitudinal axis, the first metal inlay defining a groove disposed transverse to the longitudinal axis, and the proximal end defining a stopper extending radially from the first metal inlay and defining a lateral surface;
a plastic body having a proximal area and a distal area, the proximal area disposed over the groove and connected to the first metal inlay; and
a second metal inlay disposed in the distal area of the plastic body and spaced from the first metal inlay, the second metal inlay defining an opening extending through the distal area of the plastic body along an axis disposed at an angle to the longitudinal axis and adapted to receive a locking screw;
wherein the lateral surface is aligned with and sealing against the plastic body.

15. The bone nail of claim 14, wherein the groove comprises a circumferential groove.

16. The bone nail of claim 15, wherein the groove is disposed between the proximal end and the conical section.

17. The bone nail of claim 14, wherein the first metal inlay comprises titanium.

18. The bone nail of claim 14, wherein the plastic body comprises a polymer.

19. The bone nail of claim 18, wherein the plastic body comprises a composition containing the polymer; and
wherein the polymer is selected from a group consisting of polyetherether ketones with carbon fiber reinforced polyetherether ketones and polyamides.

20. A bone nail for osteosynthesis, comprising:
a first metal inlay comprising a load-bearing component having a proximal end, a distal end, a conical section disposed between the proximal end and the distal end, and a longitudinal axis, the conical section defining a thread;
the first metal inlay defining a circumferential groove disposed transverse to the longitudinal axis and between the proximal end and the conical section;
the proximal end defining a stopper extending radially from the first metal inlay and defining a lateral surface;
a plastic body having a proximal area and a distal area, the proximal area disposed over the groove and the thread, connected to the first metal inlay, and defining a surface aligned with and sealing against the lateral surface of the stopper; and
a second metal inlay disposed in the distal area of the plastic body and spaced from the first metal inlay, the second metal inlay defining an opening extending through the distal area of the plastic body along an axis disposed at an angle to the longitudinal axis and adapted to receive a locking screw.
